(19) United States
(12) Patent Application Publication
(10) Pub. No.: US 2024/0125736 A1
(43) Pub. Date: Apr. 18, 2024

(54) CU ION SENSOR AND CU ION SENSING METHOD

(71) Applicant: Ming Chi University of Technology, New Taipei City (TW)

(72) Inventors: Sajal BIRING, New Taipei City (TW); Sheng-Chi Chen, New Taipei City (TW); Annada Sankar SADHU, New Taipei City (TW); Min-Chen Chuang, New Taipei City (TW); Kuan-Yu Chien, New Taipei City (TW)

(21) Appl. No.: 18/090,950

(22) Filed: Dec. 29, 2022

(30) Foreign Application Priority Data
Oct. 18, 2022 (TW) 111139497

(51) Int. Cl. G01N 27/622; G01N 33/18; H01L 21/02

(52) U.S. Cl. CPC G01N 27/622; G01N 33/1813; H01L 21/02186; H01L 21/02381

(57) ABSTRACT

The present invention relates to a Cu ion sensor, which greatly improves the sensitivity to Cu by a nitrogen-rich surface of a copper nitride thin film doped with a metal material. The present invention also relates to a Cu ion sensing method, in which Cu²⁺ is detected by contacting the Cu ion sensor of the present invention with the solution to be tested, and using the change in electrical conductivity of a copper nitride film doped with a metal material in the presence of Cu²⁺ in the solution.

### CROSS-REFERENCE TO RELATED APPLICATION

This application claims the priority of Taiwanese patent application No. 111139497, filed on Oct. 18, 2022, which is incorporated herewith by reference.

### BACKGROUND OF THE INVENTION

### 1. Field of the Invention

The invention relates to a Cu ion sensor and a Cu ion sensing method, in particular to a Cu ion sensor for sensing Cu ions in drinking water.

### 2. The Prior Arts

Heavy metal ions such as arsenic, chromium, copper, lead, mercury, cadmium etc. are toxic above certain contents or concentrations, which causes a threat to human health as well as environments. Therefore, removal of these bio-hazardous elements from aqueous solutions, foods, or body fluids is crucial to life. Detection of these heavy metals with high accuracy is essential prior to the removal process.

Various detection methods have been adopted in past years with respective pros and cons for specific detection of heavy metal ions. Likewise, large efforts have been dedicated to detect copper ions (Cu²⁺), since it is an essential trace element to life while it becomes deadly over a concentration of 1.3 mg/L.

Several predominant analytical processes such as colorimetry, atomic absorption spectroscopy, inductively coupled plasma atomic emission spectrometry, electrochemical, and optical chemical sensing are generally carried out for detection of Cu²⁺. Although the above detection methods can reach nano-molar to pico-molar levels, the instruments are complicated to operate, highly specialized, expensive, and difficult to maintain.

Therefore, in the present, there is an urgent need to develop a novel Cu ion sensor, which not only has high sensitivity, low detection limit and low cost, but also can achieve simple process and simple measurement technology.

### SUMMARY OF THE INVENTION

In view of this, the present invention provides a Cu ion sensor and a Cu ion sensing method.

The Cu ion sensor of the present invention comprises: a substrate; a patterned electrode disposed on the substrate; and a copper nitride film, which is disposed on the patterned electrode and the substrate and doped with a metal material, wherein a nitrogen-rich plane of the copper nitride film increases with an increase in a doping content of the metal material without exceeding a maximum doping content of the metal material.

Specifically, the substrate can be a quartz glass substrate; the patterned electrode can be an indium-tin-oxide (ITO) electrode, and further can be an interdigitated electrode; the copper nitride film can be a Cu₃N film; and the metal material can be Ti.

In addition, the present invention provides a Cu ion sensing method comprising the following steps: providing a solution to be tested containing Cu ions; contacting the Cu ion sensor with the solution to be tested; applying a fixed bias to the Cu ion sensor; measuring a current of the solution to be tested; using the current to calculate an electrical conductivity of the solution to be tested; and plotting the electrical conductivity against a Cu ion concentration to obtain a linear relationship to detect the Cu ions.

In addition, as the content of the metal material in the copper nitride film increases, the Cu ion sensor of the present invention has high sensitivity and high response to Cu, so it is suitable for detecting Cu²⁺ at concentrations in pico-molar to nano-molar levels.

The effects of the present invention are not limited to the above-mentioned effects, and those skilled in the art can clearly understand other non-mentioned effects from the description of the claims.

### BRIEF DESCRIPTION OF THE DRAWINGS

Those skilled in the art will understand the above and other objects, features and advantages of the present invention more clearly by the description of the exemplary embodiments in detail with reference to the accompanying drawings, wherein:

FIG. 1A is a schematic top view showing a Cu ion sensor according to an embodiment of the present invention;

FIG. 1B is a schematic cross-sectional view showing the Cu ion sensor according to the embodiment of the present invention;

FIG. 1C is an optical image of the Cu ion sensor showing a copper nitride thin film on parallel ITO electrode strips;

FIG. 2 shows deposition rates of the copper nitride films according to the embodiments of the present invention under different DC powers;

FIG. 3 shows EPMA results of the copper nitride films according to the embodiments of the present invention under different DC powers;

FIG. 4 shows XRD patterns of the copper nitride films of the embodiments of the present invention with different Ti contents;

FIG. 5A shows the relationship between the electrical conductivity of the copper nitride films of the embodiments of the present invention with different Ti contents and the Cu²⁺ concentration;

FIG. 5B shows the response of the copper nitride films of the embodiments of the present invention with different Ti contents to Cu²⁺ concentration;

FIG. 6 shows XRD patterns of the copper nitride film of the embodiment of the present invention doped with higher Ti content;

FIG. 7 shows the response of a Cu ion sensor of an embodiment of the present invention to different metal ions in drinking water;

FIG. 8 shows the response of the Cu ion sensor of the embodiment of the present invention to the Cu²⁺ concentration in the PBS solution;

FIG. 9 shows the response of the Cu ion sensor of the embodiment of the present invention to the Cu²⁺ concentration in drinking water; and

FIG. 10 shows the flowchart of the Cu ion sensing method of an embodiment of the present invention.

### DETAILED DESCRIPTION OF THE PREFERRED EMBODIMENT

Advantages and features of the present invention and implementation methods thereof will be more clearly understood according to the embodiments described below with reference to the accompanying drawings. However, the present invention is not limited to the following embodiments, but can be implemented in various forms.

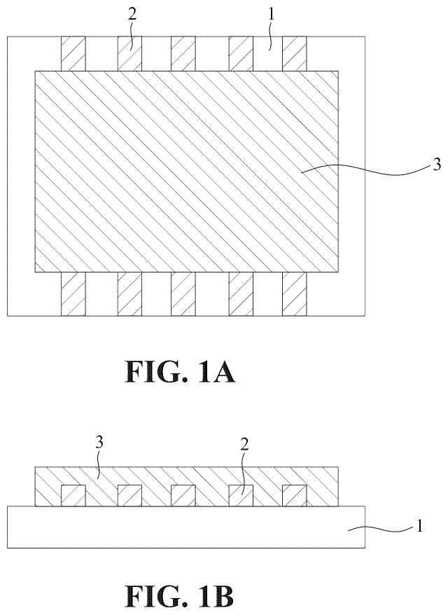

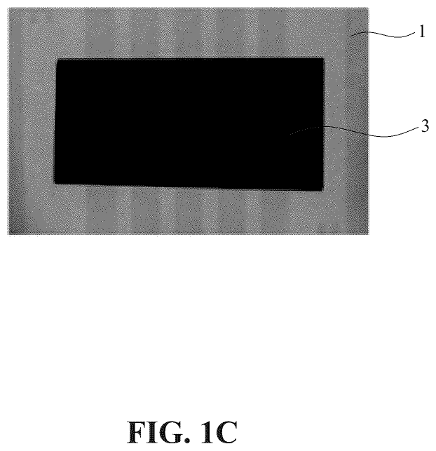

Referring to FIGS. 1A to 1C, a Cu ion sensor according to an embodiment of the present invention includes a substrate 1, a patterned electrode 2, and a copper nitride film 3.

The substrate 1 is, for example, but not limited to, a quartz glass substrate, available from Corning, such as an EAGLE XG® glass substrate.

The patterned electrode 2 is, for example, but not limited to, an indium-tin-oxide (ITO) electrode, available from Sanyo, Japan.

The patterned electrodes 2 further include interdigitated electrodes.

The copper nitride film 3 is doped with a metal material, wherein the copper nitride can be Cu₃N, and the metal material can be titanium (Ti).

Copper and titanium targets with high purity (4N) were purchased from Ultimate material technology (UMAT). The Cu target was 3 inch in diameter and 6 mm in thickness. The Ti target was 2 inch in diameter and 6 mm in thickness.

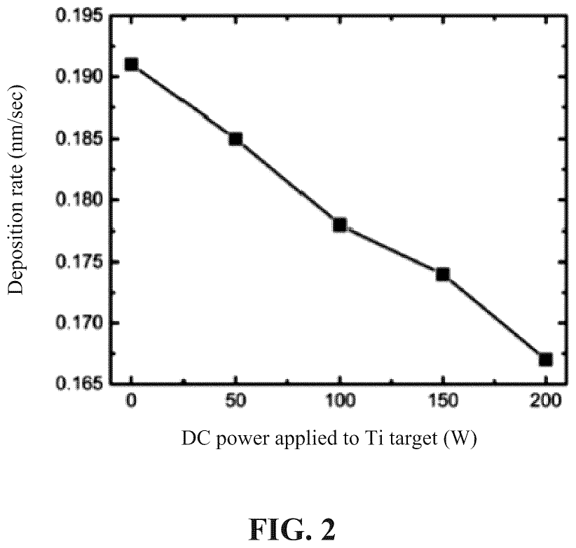

FIG. 2 shows deposition rates of the copper nitride films according to the embodiments of the present invention under different DC powers.

Referring to FIG. 2, a 200 nm of Ti-doped Cu₃N thin film was deposited on ITO-coated quartz glass substrate by High Power Impulse Magnetron Sputtering (HiPIMS). The Ti doping amount in the Cu₃N was controlled by applying various DC power to the Ti sputtering source (Ti source).

The deposition rate of the Cu₃N film was monitored at different DC powers. The deposition rate of pure Cu₃N film (i.e., the DC power applied to the Ti source was zero) was 0.191 nm/s and decreased monotonically with the increase in the DC power applied to the Ti source.

As the power applied to Ti increases, more Ti atoms are sputtered, resulting in a shortening of the mean free path and an increase in probability of collisions among atoms. Therefore, the atoms lose energy, which is manifested as the lowered deposition rate with the increase in DC power applied to the Ti source. The film deposition rate was reduced to 0.166 nm/s at a DC power of 200 W.

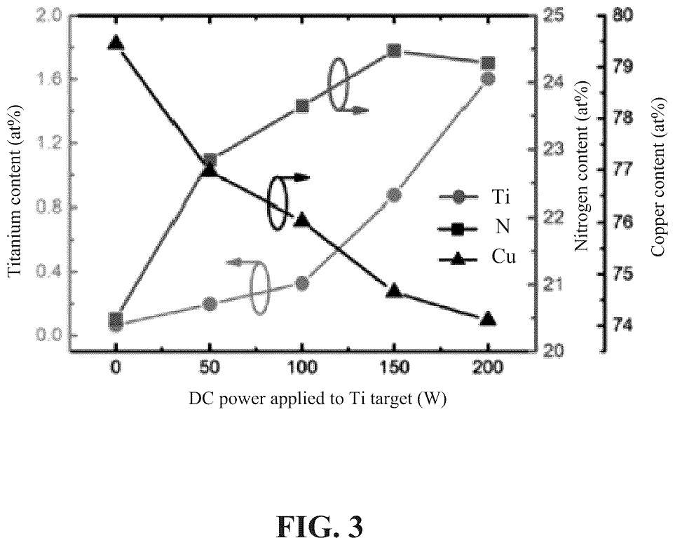

FIG. 3 shows EPMA results of the copper nitride films according to the embodiments of the present invention under different DC powers.

Referring to FIG. 3, five different Ti-doped Cu₃N films, corresponding to DC power of 0 W, 50 W, 100 W, 150 W, and 200 W, respectively, were fabricated in the present invention.

The growth composition of the films was studied by electron probe microanalyzer (EPMA) and is shown in FIG. 3. The Ti content in the Cu₃N film increases monotonically with the increase in the DC power, in which the Ti content in the Cu₃N film increases slowly at low power and increases rapidly at higher power. The Cu content follows the reverse trend of that of the Ti content in the film, i.e., the Cu content decreases with the increase in the DC power applied to the Ti source. The Cu content decreases with a faster rate at low DC power and then decreases with a relatively slower rate at higher DC power. Finally, the nitrogen content increases with the increase in the DC power, and almost levels off at high powers. The EPMA results indicate that five Ti-doped Cu₃N films with Ti contents of 0, 0.19, 0.33, 0.87, and 1.6 at % have been fabricated in the present invention.

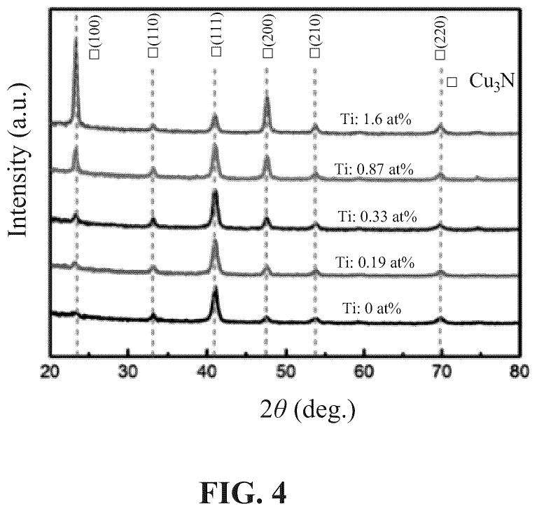

FIG. 4 shows XRD patterns of the copper nitride films of the embodiments of the present invention with different Ti contents.

To investigate the crystal nature of these five Ti-doped Cu₃N films, XRD (X Ray Diffractometer) analysis is carried out in the present invention. The XRD patterns of all these films are shown in FIG. 4. The Cu₃N films with a Ti content of 0 at % reveals the XRD pattern of a pure Cu₃N lattice with a prominent peak at 2θ=41° corresponding to (111) plane without showing any presence of Ti element. The XRD peak intensity corresponding to the (111) plane decreases gradually with the increase in Ti content in the Cu₃N film. The peak intensity decreases much faster at higher Ti content. On the other hand, the peak intensities corresponding to (100) and (200) planes of the Cu₃N lattice increase with the increase in Ti content. While the increase in the XRD peak intensity corresponding to the (200) plane is almost linear, the peak intensity of the (100) plane increases more slowly at low Ti content and increases faster at higher Ti content.

The XRD data indicates that the (100) plane dominates over other Cu₃N planes at a Ti content of 1.6 at %. The (100) and (111) planes of Cu₃N lattice suggest the nitrogen-rich and copper-rich surfaces, respectively. When more nitrogen atoms are presented on the surface of Cu₃N film, the probability to capture Cu ions by π-π intermolecular interactions between nitrogen atom and Cu ions increases, suggesting higher sensitivity to Cu ions. Therefore, Cu₃N film with higher Ti content is supposed to be more sensitive to Cu ions.

It is noted that the full width at half-maximum (FWHM) of the peaks corresponding to the (100) and (200) planes decreases with the increase in Ti content, suggesting that the films with high Ti content have higher crystallinity Therefore, the XRD data suggests the successful fabrication of pure Cu₃N film and Ti-doped Cu₃N film with high quality.

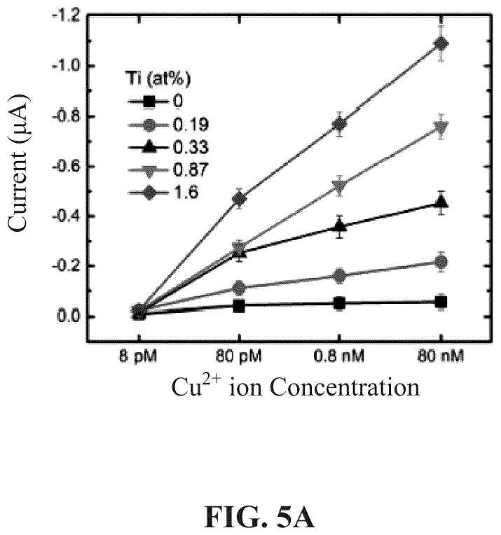

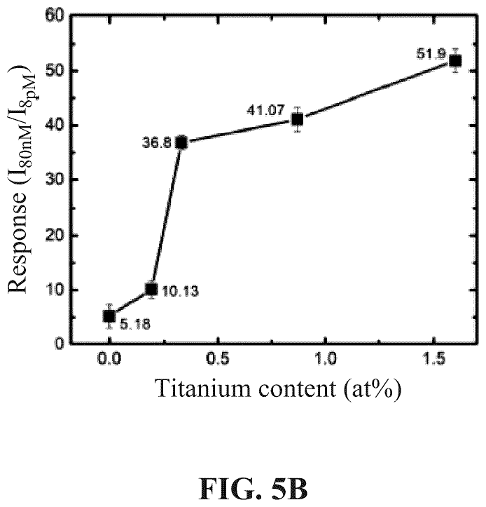

FIG. 5A shows the relationship between the electrical conductivity of the copper nitride films of the embodiments of the present invention with different Ti contents and the Cu²⁺ concentration.

When applying different concentration of Cu²⁺, the electrical conductivity of the Ti-doped Cu₃N films will change. As depicted in the FIG. 5A, under the applied bias of 4 V, the change in current over the Cu²⁺ concentration increases rapidly as the doping of Ti in Cu₃N films increases. According to the XRD patterns, the nitrogen-rich (100) plane of the Cu₃N film increases with the increase in the Ti content. When there are more nitrogens on the surface of the film, it is beneficial to enhance π-π intermolecular bonding with copper ions, resulting in improved electrical conductivity. Consequently, the Cu₃N film with a Ti doping content of 1.6 at % shows the highest electrical conductivity to Cu²⁺.

FIG. 5B shows the response of the copper nitride films of embodiments of the present invention with different Ti contents to Cu²⁺ concentration.

As shown in FIG. 5B, the response (I_{80 nM}/I_{8 pM}) to Cu²⁺ concentration of the Ti-doped Cu₃N films of the present invention with different Ti contents at an applied bias of 4 V is plotted herein. It is obvious that the response increases significantly to a value of 51.9 with the increase in Ti content in Cu₃N film. Consequently, the Cu₃N film with a Ti doping content of 1.6 at % shows the highest response to Cu²⁺. This high response shows that the proposed device is suitable for sensing Cu²⁺ at concentrations in pico-molar to nano-molar levels.

The efficient sensing at such a low concentration (pico-molar) imparts the proposed Cu ion sensor in the present invention an excellent industrial applicability.

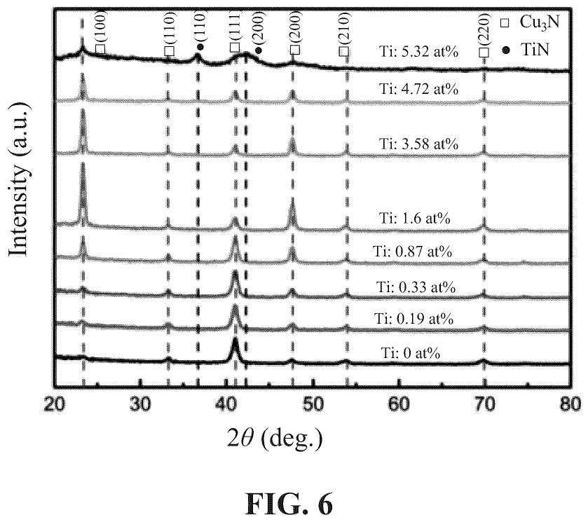

It is intuitive to increase the Ti content to achieve higher response of the proposed sensor. However, as shown in FIG. 6, three other Ti-doped Cu₃N thin films with Ti contents of 3.58 at %, 4.72 at %, and 5.32 at %, respectively, are further fabricated in the present invention.

The XRD data shows that when the Ti content in the Cu₃N film is higher than 1.6%, the formation of TiN phase results in gradual decreases of the peak intensities corresponding to the (100) and (200) planes of Cu₃N phase. Since the nitrogen-rich planes (100) and (200) of Cu₃N are responsible for the Cu²⁺ sensing, it can be known that 1.6 at % of Ti is the maximum allowed doping amount in the Cu₃N film.

These Ti-doped Cu₃N films are then applied to fabricate Cu²⁺ sensor. Ti-doped Cu₃N films with a thickness of 200 nm are deposited on patterned ITO on glass substrates. Plan and cross-sectional views and optical images of the Cu ion sensor are schematically presented in FIGS. 1A to 1C. The ITO electrode strips with a width of 200 μm and a gap of 100 μm are used as consecutive electrodes in the conductometric measurements.

The Cu ion sensor of the present invention can be used to detect the concentration of Cu ions in drinking water and body fluids. The body fluids can be, for example, urine, blood, sweat, and tears.

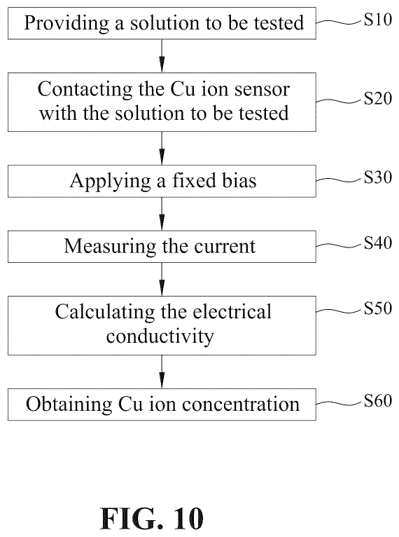

An embodiment of the present invention further provides a Cu ion sensing method, using the Cu ion sensor described above for measurement, as shown in FIG. 10, the method includes the following steps:
S10: providing a solution to be tested containing Cu ions;
S20: contacting the Cu ion sensor with the solution to be tested;
S30: applying a fixed bias to the Cu ion sensor;
S40: measuring a current of the solution to be tested;
S50: using the current to calculate an electrical conductivity of the solution to be tested; and
S60: plotting the electrical conductivity against a Cu ion concentration to obtain a linear relationship to detect the Cu ions.

In step S10, the solution to be tested containing Cu ions may be drinking water and phosphate buffered saline (PBS) solution. In step S20, the sensitivity of the proposed sensor is measured according to the change in the current-voltage (I-V) characteristics of the two consecutive ITO electrodes in presence or absence of Cu²⁺ solution on the Cu₃N films.

In step S60, the current increases almost linearly with the increase in the Cu²⁺ concentration ranging from 8 pM to 80 nM. The high concentration of HNO₃ in the Cu²⁺ solution erodes the Cu₃N film, severely limiting the maximum concentration as 80 nM. This long dynamic range (80 nM/8 pM=4 orders of magnitude) of Cu ion sensing is highly promising in industrial or medical fields.

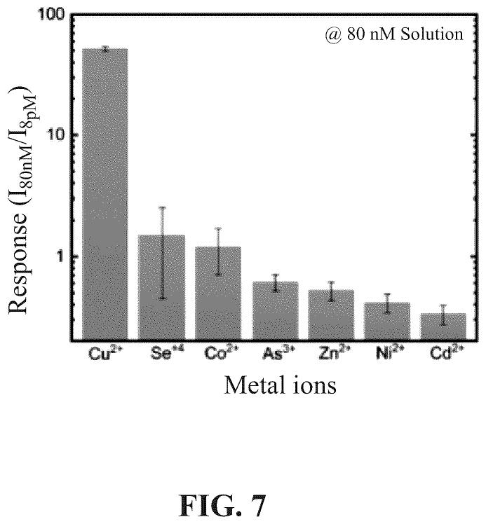

FIG. 7 shows the response of a Cu ion sensor of an embodiment of the present invention to different metal ions in drinking water.

To test the selectivity of the proposed Cu ion sensor, solutions of few other metal ions, such as Selenium (Se⁴⁺), Cobalt (Co²⁺), Arsenic (As³⁺), Zinc (Zn²⁺), Nickel (Ni²⁺), and Cadmium (Cd²⁺) which are found in drinking water in general, are prepared therein.

In FIG. 7, the responses of all these metal ions are compared with that of the Cu ions. It is obvious that the response to the Cu ions is much prominent compared to those to other metal ions in the solution.

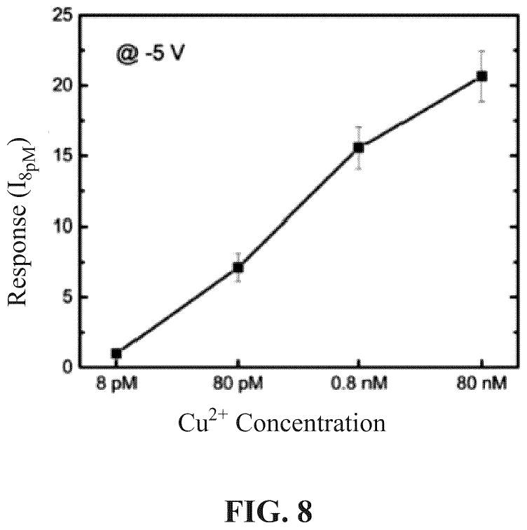

FIG. 8 shows the response of the Cu ion sensor of the embodiment of the present invention to the Cu²⁺ concentration in the PBS solution.

To test the feasibility of the proposed sensor for practical application, Cu ions are applied in the PBS solution and the responses are measured.

The sensor shows a significant response to Cu²⁺ in the PBS solution containing disodium hydrogen phosphate, sodium chloride, potassium chloride, and potassium dihydrogen phosphate.

In addition, the sensing of Cu ion in practical sample (i.e., tap water with unknown composition) is tested in present embodiment. Cu ions are added in the tap water which has been filtered by reverse-osmosis (RO) to remove bigger particles.

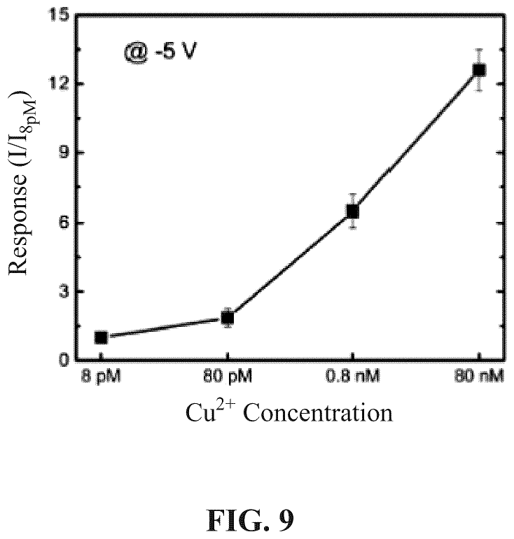

FIG. 9 shows the response of the Cu ion sensor of the embodiment of the present invention to the Cu²⁺ concentration in drinking water.

FIG. 9 depicts the response of the sensor when different concentrations of Cu ions are added into the tap water. This data shows a significant response to Cu ions, specifically, Cu ions can be detected in the range of 8 pM to 80 nM, so the proposed Cu ion sensor in the present invention is capable of detecting Cu ions in the range of 8 pM to 80 nM in drinking water and thus opens up an avenue to monitoring drinking water in residential and commercial zones.

Cu ion concentrations in any chemical solutions (industrial or domestic wastewater) can be measured with the proposed Cu ion sensors provided the compatibility of the solution to be tested matches with the features of the Cu ion sensor. For example, Cu ion concentrations in chemical solutions containing organic solvents or being acidic or basic can be measured if the compatibility of these chemical solutions matches with the features of the Cu ion sensor.

In conclusion, a Cu ion sensor composed of a copper nitride film doped with metal material deposited on a patterned electrode on a substrate has successfully demonstrated that it can reach pico-molar level detection of Cu ions. With the increase in the doping content of the metal materials, the nitrogen-rich (100) plane is more than the copper-rich (111) plane of the copper nitride film, which can improve the highest response of the copper nitride film to Cu ions. Therefore, due to the high sensitivity of the Cu ion sensor of the present invention to Cu, Cu ions in various solutions can be effectively detected.

Although the embodiments of the present invention have been described in more detail with reference to the accompanying drawings, the present invention is not limited thereto, and therefore, it should be understood that the above-described embodiments are illustrative in all aspects and do not limit the present invention.

What is claimed is:

1. A Cu ion sensor, comprising:
a substrate;
a patterned electrode disposed on the substrate; and
a copper nitride film, which is disposed on the patterned electrode and the substrate and doped with a metal material, wherein a nitrogen-rich plane of the copper nitride film increases with an increase in a doping content of the metal material without exceeding a maximum doping content of the metal material.

2. The Cu ion sensor of claim 1, wherein the substrate is a quartz glass substrate.

3. The Cu ion sensor of claim 1, wherein the patterned electrode is an indium-tin-oxide (ITO) electrode.

4. The Cu ion sensor of claim 3, wherein the patterned electrode is an interdigitated electrode.

5. The Cu ion sensor of claim 1, wherein the copper nitride film is a Cu₃N film, and a (100) plane of a lattice thereof is the nitrogen-rich plane.

6. The Cu ion sensor of claim 5, wherein as the nitrogen-rich plane of the Cu₃N film increases, a bonding between nitrogen atoms of the Cu₃N film and Cu ions to be detected is enhanced, so that the Cu ion sensor has a detection limit of 8 pM for a Cu ion concentration.

7. The Cu ion sensor of claim 1, wherein the metal material is titanium.

8. The Cu ion sensor of claim 7, wherein the maximum doping content of the metal material is 1.6 at %.

9. A Cu ion sensing method, comprising the following steps:
providing a solution to be tested containing Cu ions;
contacting the Cu ion sensor of claim 1 with the solution to be tested;
applying a fixed bias to the Cu ion sensor;
measuring a current of the solution to be tested;
using the current to calculate an electrical conductivity of the solution to be tested; and
plotting the electrical conductivity against a Cu ion concentration to obtain a linear relationship to detect the Cu ions.

10. The Cu ion sensing method of claim 9, wherein the linear relationship is exhibited when the Cu ion concentration is 8 pM to 80 nM.

11. The Cu ion sensing method of claim 9, wherein the solution to be tested is body fluid.

12. The Cu ion sensing method of claim 9, wherein the solution to be tested is drinking water, and the Cu ion concentration is detectable in a range of 8 pM to 80 nM.

13. The Cu ion sensing method of claim 9, wherein the solution to be tested is chemical solution.
